(19) United States
(12) Patent Application Publication
(10) Pub. No.: US 2005/0130241 A1
(43) Pub. Date: Jun. 16, 2005

(54) Immunological detection of prostate diseases and prostasome-related conditions

(76) Inventors: Lena Carlsson, Uppsala (SE); Ove Nilsson, Uppsala (SE); Gunnar Ronquist, Uppsala (SE); Anders Larsson, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 10/468,949

(22) PCT Filed: Feb. 22, 2002
(86) PCT No.: PCT/SE02/00313

(30) Foreign Application Priority Data
Feb. 22, 2001 (SE) 0100595.8

(51) Int. Cl. G01N 33/53; G01N 33/537; G01N 33/543; C07K 16/42

(52) U.S. Cl. 435/7.92; 530/387.2

(57) ABSTRACT

The present invention relates to a diagnostic and/or prognostic reagent comprising a component which selectively binds to anti-prostasome autoantibodies, as well as an immunoassay using said reagent. Furthermore, the invention relates to an in vitro method for diagnosing and/or prognosticating a condition reflecting the prostasome presence in body fluids, comprising a) binding of anti-prostasome autoantibodies with a component which selectively binds to anti-prostasome autoantibodies; and b) detection of said binding. The conditions may be different forms of prostate cancer or prostasome-related diseases or other conditions where anti-prostasome autoantibodies are present in the body.

### FIELD OF THE INVENTION

The present invention relates to immunological detection of prostate diseases, among which prostate cancer holds a unique position, and of prostasome-related diseases with examples on clinical applications of the invention.

### BACKGROUND OF THE INVENTION

Prostate cancer is the fourth most commonly diagnosed cancer in men worldwide and the most commonly diagnosed in Swedish men. The age-adjusted incidence of prostate cancer has increased by about 1.8% per year over the past decade. This increase may be partly due to the introduction of better diagnostic and therapeutic techniques. The prevalence of prostate cancer is comparatively low in men younger than 60 years in Sweden but is 4% in men 75-79 years of age and is as high as 5-6% in men over the age of 80 years. Autopsy studies have indicated a much higher prevalence of latent prostate cancer. Hence, a prevalence of 44% was found in men 50-59 years of age and 83% in those 70-79 years, which suggests that many prostate cancers do not reach a clinically significant stage. In Sweden, 6,610 new cases of prostate cancer were reported in 1998; 67% of these men were older than 70 years, and only 0.26% were younger than 50 years. In 1997, 2,448 men died of prostate cancer indicating substantial mortality from this cancer form. It has also been shown that the younger the patient is at the time of diagnosis, the higher is the mortality. The causes of prostate cancer are essentially unknown, although several factors have been shown to be associated with a higher risk for this type of cancer as increasing age, family history of prostate cancer, men in Western countries, and especially American men of African heritage.

Nonbacterial prostatitis is a benign and common disease mostly among young men. The etiology of the disease is not fully understood. The symptoms include genital and pelvic pain, urgency to void and cold sensitivity. Although the symptoms are suggestive of an infection in the prostate gland, there is a lack of clear bacterial infectious etiology for a majority of men with these symptoms. Among alternative etiologic factors, it has been claimed that an autoimmune component to the disease might exist. This means that a self-reactivity directed against the prostate or a prostate component by the immune system could be involved in the etiology of the disease. Accordingly, a production of autoantibodies against some component of the prostatic secretion in patients with nonbacterial prostatitis could contribute to their symptoms.

Autoantibodies can be produced in the body and cause severe diseases, like rheumatic artritis and a type of diabetes, by attacking cells carrying the corresponding antigens. No autoantibodies, which recognize or affect cancer metastases, have yet been reported.

The prostate gland is one of the major accessory genital glands with mainly exocrine functions. Although being a unified organ, three pairs of lobes can be distinguished. Histologically the prostate is composed of a large series of independent branching ducts, all of which enter the prostatic urethra. Hence, the prostate gland and its secretion represent a closed secretory system and the secretory components will normally not be able to appear in the circulation. Human prostatic fluid contains high amounts of monovalent and divalent cations. It is also rich in enzymes involved in carbohydrate metabolism and protein degradation. Besides these soluble substances the prostate gland secretes the above-mentioned prostasomes which are prostatic secretory products coating the sperm cells.

Prostasomes are surrounded by a bilayered and sometimes tri- or multi-layered membrane and they have a diameter of 40-500 nm. Prostasomes are secretory products of the prostate gland. The membrane architecture of these organelles is complex and two-dimensional gel electrophoresis of membrane material has revealed about 80 different protein entities. Also, an unusually high cholesterol/phospholipid ratio is inherent in this membrane. The prostasomes contain neuroendocrine and CD molecules and many different enzymes are part of the prostasome membrane mosaic. Prostasomes have been ascribed many different biologic activities, but their physiologic function is still unclear. They can interact with spermatozoa and promote their motility characteristics in different ways. They are also immunosuppressive and inhibit superoxide anion generation by neutrophil granulocytes. The prostasomes can modulate complement-mediated immune responses, and CD 59, an inhibitor of the membrane attack complex of complement, resides on prostasomes.

The present inventors have previously isolated and purified prostasomes not only from seminal plasma and expressed prostatic fluid (exprimate) but also from the human prostate gland as well as from vertebrate metastases of prostatic cancer. They have also cultured human prostatic cancer cells of the PC3 and similar cell lines on plates in monolayer and found that they can produce prostasomes. These PC3 cells have been fractionated and the prostasomes purified C3 prostasomes). In addition, the present inventors and others have produced monoclonal (1, 3-4) and polyclonal (2) antibodies against some types of prostasomes.

Historically, prostate cancer was most often diagnosed in men presenting with symptoms derived from a local tumour or metastatic spread of a tumour, such as dysfunctional voiding or bone pain, and the disease was at an advanced stage at the time of diagnosis. Occasionally, it was an accidental finding on digital rectal examination or upon histological examination of tissue obtained during surgery on men with benign prostatic hyperplasia. Accordingly, there is a need of improvement and intensified use of diagnostic procedures on men at risk, resulting in more extensive detection of nonlethal prostate cancers.

Measurement of prostate specific antigen (PSA) in serum was initiated in the latter half of the 1980s in many Western countries. This measure changed the pattern of diagnosis of prostate cancer with more cases detected at an early stage and fewer cases at advanced stages. However, since PSA is not a prostate cancer specific marker in serum it is not the ideal diagnostic marker and therefore not accommodated for screening of prostate cancer. There is growing concern about the PSA-test, as it is employed at present. The test is hampered by its incapacity for discriminating in a proper way between benign prostatic hyperplasia and prostate cancer and, what is more, between prostate cancer with high metastasising potential (aggressive prostate cancer) and such cancer with no or weak aggressiveness. This inability of the test often will end up in truncating surgery, i.e. total prostatectomy. This type of overtreatment is a great inconvenience not only for patients but also for society due to extra costs.

### SUMMARY OF THE INVENTION

The present invention is based upon the demonstration of anti-prostasome autoantibodies in serum of patients with prostate cancer due to the border-breaking growth of neoplastic cells in the prostate. The invention also comprises the idea that prostasomes, due to their smallness, will appear in the circulation earlier than the much bigger (about 150 times) prostate cancer cells, which are spread via the blood. Accordingly, a time-window may be offered for the prostasomes, during which the anti-prostasome autoantibodies can be developed and detected by our test, before the bigger prostate cancer cells will be released into circulation and set their metastases. The anti-prostasome autoantibodies in body fluids (here represented by blood plasma/serum) may, for example, be detected with an ELISA technique (see below). Herewith, the invention presents a new way for diagnosing early metastasis and for discriminating between the dangerous (metastasis-prone) prostate cancer and the more or less harmless (not metastasis-prone) variant of prostate cancer.

The anti-prostasome autoantibodies, which we have detected in serum from patients with a prostate cancer, do not attack the cancer cells and are therefore not recognized clinically.

In a first aspect, the invention relates to an agent which selectively binds to anti-prostasome autoantibodies. In one embodiment, the agent is a diagnostic and/or prognostic reagent comprising a component which selectively binds to anti-prostasome autoantibodies. The component should comprise structures involved in the binding of prostasomes and anti-prostasome autoantibodies, for example prostasomes or epitope(s) exposed in prostasomes binding to anti-prostasome antibodies, i.e. with selective affinity to anti-prostasome autoantibodies. By the term ‘selective’ is also meant that the binding is mainly selective. The reagent (i.e. antigen) may comprise prostasomal-like material, for example, of natural, pathological, synthetic or recombinant origin.

In a second aspect, the invention relates to a kit for diagnosis/prognosis of prostasome-related diseases comprising a reagent as defined above.

In a third aspect, the invention relates to an immunoassay for detection of anti-prostasome autoantibodies in body fluids comprising the above reagent. The body fluid may be serum or plasma. Further alternatives are urine and semen.

Optionally, the reagent (i.e. the antigen) is bound to a solid support, such as a microtitre plate which is commonly used in, for example, ELISA. The immunoassay according to the invention may be combined with a conventional PSA assay if desired.

In a fourth aspect, the invention relates to an in vitro method for diagnosing and/or prognosticating a condition reflecting the prostasome presence in body fluids, comprising
a) binding of anti-prostasome autoantibodies with a component which selectively binds to anti-prostasome autoantibodies; and
b) detection of said binding. Preferably, the detection is by by ELISA or flow cytometry techniques.

The condition may be cancer, such as prostate cancer, or prostatic diseases. For instance, the demonstration of anti-prostasome autoantibodies in body fluids may be important to better differentiate between bacterial and nonbacterial prostatitis (see above). An improved differential diagnosis between these two conditions would facilitate the position that should be taken to the question of proper treatment.

In a fifth aspect, the invention relates to use, or method of using, of an agent which selectively binds to anti-prostasome autoantibodies for the production of a drug for prevention and/or treatment of prostasome-related diseases. For example, the drug may be formulated as a vaccine.

### DETAILED DESCRIPTION OF THE INVENTION

The invention will now be described more closely below in association with a non-limiting experimental part. The following procedures will be exemplified:
1. Preparation of prostasomes
2. Production of antibodies against prostasome components
3. Characterisation of prostasomes from seminal plasma, prostate tissues, prostate cancers, and PC3 cells
4. Functional similarities between seminal prostasomes and PC3 prostasomes
5. Anti-prostasome autoantibodies in serum from patients with metastasis of prostate cancer
6. Anti-prostasome autoantibodies in serum from patients with urological symptoms.

Prostasomes were prepared from different sources, namely from seminal plasma, prostate tissues, bone metastases of prostate cancers, and the cancer cell line PC3 (p.1). Polyclonal chicken antibodies and monoclonal mouse antibodies were produced against some of these preparations (p.2). The monoclonal antibodies, which were directed against various components of the prostasomes, were applied to characterise differences among the types of prostasome (p.3). In addition, the differential expression of, for instance, enzymes and CD-factors were studied (p.3). Also functional properties, for instance, the prostasome ability to activate sperm cells were compared between prostasome types (p.4).

The useful application of prostasome components in clinical practice was examined both for immunodiagnosis of metastasis of prostate cancer (p.5) and for immunodetection of anti-prostasome autoantibodies among patients with urological disturbances (p.6).

1. Preparation of Prostasomes

Seminal plasma: Semen samples were centrifuged for 20 minutes at 1 000×g to separate spermatozoa and other cells from the seminal plasma, which was pooled (12-15 samples) and ultracentrifuged at 10 000×g for 15 minutes to pellet possible cells and cell debris. The supernatant was subsequently subjected to another ultracentrifugation for 2 hours at 100 000×g to pellet the prostasomes. The prostasomes were resuspended in 30 mmol/L Tris-HCl, 130 mmol/L NaCl, pH 7.6 (isotonic Tris-HCl buffer). The pellet suspension was further purified on a Sephadex G 200 column (Pharmacia AB, Uppsala, Sweden), to separate them from an amorphous substance. The eluant was the isotonic Tris-HCl buffer, and the eluate was monitored at 260 and 280 nm. Those fractions (5-12) with initial elevated UV absorbance were pooled and ultracentrifuged at 100 000×g for 2 hours and diluted to a protein concentration of 2 mg/ml using a Protein Assay ESL method (Boehringer Mannheim, Germany).

Prostate tissues and bone metastases: Tissues from two prostate glands and 12 bone metastases of prostate cancers (PAD verified) were homogenised in the isotonic Tris-HCl buffer. After homogenisation, the suspensions were first centrifuged 3 times at 3000×g, +4° C. for 15 minutes, then twice for 15 minutes at 10 000×g, +4° C. The supernatants obtained were then ultracentrifuged for 2 hours at 100 000×g. The pellets obtained were thereafter treated exactly in the same way as the seminal plasma samples described above.

PC-3 cell line: The human prostatic carcinoma cell line PC-3 was obtained from the American Type Culture Collection (Rockville, Md., USA). The cells were maintained in RPMI 1640 cell culture medium supplemented with 10% heat-inactivated fetal calf serum and 2 mM L-glutamine (Sigma Chemicals, MO, USA.). The cells were grown in Falcon Petri dishes (100 mm) at 37° C. and each plate yielded 2-3×10⁶ cells, which were removed by trypsin and carefully washed in the isotonic Tris-HCl buffer and centrifuged. They were again suspended in the Tris-HCl buffer and frozen at −70° C. The frozen PC-3 cells from about 8-10 plates were thawed and pooled and the suspension of disintegrated cells was centrifuged at 1500×g for 30 minutes and then at 10 000×g for 15 minutes to remove cell debris. The supernatant was ultracentrifuged at 100 000×g for 2 hours, and pelleted prostasomes were suspended in the isotonic Tris-HCl buffer. The dissociated prostasomes were run through the Sephadex G 200 column and treated as above.

2. Production of Antibodies Against Prostasome Components

The immunogenicity of the prostasomes has been demonstrated by the production of several types of antibodies against prostasomes from two different sources using both mouse and hen as hosts. Monoclonal antibodies against seminal prostasomes were produced by intrasplenic immunization. One μg of purified prostasomes was injected four times in the spleens of mice. Hybridomas were tested in an ELISA system, using seminal prostasomes as the coating antigen.

Chicken polyclonal antibodies against prostasomes were produced by immunisation with seminal prostasomes and cancer prostasomes, respectively.

3. Characterisation of Prostasome from Seminal Plasma, Prostate Tissues, Prostate Cancer Metastases and PC3 Cells

The prostasome membrane is composite and 2-dimensional electrophoresis has revealed at least 80 different protein entities. Determination of some of the proteins associated with prostasomes has been done in an attempt to characterise or differentiate the prostasomes of the four different sources, seminal plasma, prostate tissue, prostate cancer metastases and PC3 cells. Results: Aminopeptidase (CD13), Dipeptidylpeptidase IV (CD26), the neuropeptides: Chromogranin A and B, Neuropeptide Y (NPY) and Vasoactiveintestinal peptide (VIP) are all present in high amounts in seminal prostasomes. They also appear in prostate tissue prostasomes, CD 26, Chromogranin B and VIP at equal amounts as in the seminal prostasomes, but only at about 50% of the Chromogranin A, 30% of CD13 and 4% of NPY compared to the concentrations in seminal prostasomes.

Prostasomes derived from the prostate cancer metastases and PC3 cell line showed a somewhat different pattern with the Chromogranin A concentration being 3-5 times higher than in seminal prostasomes and NPY, VIP and CD13 less than 10% of the seminal prostasomes.

Some differences in protein pattern between the four prostasome types could also be demonstrated by electrophoresis on SDS-PAGE.

4. Functional Similarities Between Seminal Prostasomes and PC3 Prostasomes

Prostasome-like granules are present in the PC3 prostate cancer cells. Since the seminal prostasomes are able to promote the forward motility of human spermatozoa, we conducted a study to determine whether PC3 prostasomes exerted similar effects to those of seminal prostasomes on buffer-washed spermatozoa from normospermic semen samples.

Semen samples were obtained from normospermic men, according to WHO laboratory manual, during evaluation for in vitro fertilisation. Motile spermatozoa were obtained by a swim-up procedure. Prostasomes were obtained from the human prostatic carcinoma cell line PC-3 and purified according to our protocol.

The sperm motility analysis was done in accordance with the guidelines for application of CASA technology. At each measurement time, at least 200 spermatozoa from each aliquot sample were analysed in order to monitor sperm movement characteristics. This was done with an HTM semi-automated motility analyser (Hamilton-Thorn Research. Inc., Danvers, Mass., USA). The effects of PC3 prostasomes and seminal prostasomes on the sperm cell motility over time were compared at a protein concentration of 0.1 mg/ml. There were no significant differences between the two types of prostasomes in their stimulatory ability.

We conclude that PC3 prostasomes, isolated from in vitro-grown PC3 cells, bear a functional resemblance to prostasomes isolated from human seminal plasma.

5. Anti-Prostasome Autoantibodies in Serum from Patients with Metastasis of Prostate Cancer

Serum samples from 13 men with PAD verified prostate cancer with metastases were included in the study. As control group we used healthy blood donors, 20 men and 20 women, age 20-40 years which were all tested for low PSA values.

An immunoassay was designed to detect anti-prostasome autoantibodies in serum. For enzyme-linked immunosorbent assay (ELISA), plates were coated (F96 Polysorp, Nunc) with 4 μg purified prostasomes obtained from prostate tissue (see p.1) diluted in 100 mmol/L NaHCO₃, pH 9.5 (coating buffer) for 2 hours at 37° C. The plates were washed and blocked for one hour, 37° C., with the coating buffer containing 3% BSA. After blocking, the plates were washed 3 times with 200 μl phosphate buffered saline with 0.1% Tween (PBS-T) and then incubated with 200, patient sera (dilution 1:50 in PBS) for 2 hours, 37° C.

After 3 new washes with 200 μl PBS-T, 100 μl goat-anti-human IgG-horse radish peroxidase (HRP) conjugated antibodies were added (dilution 1:1000 in PBS) and incubated for 1 hour at room temperature. The plates were washed 3 times with 200 μl PBS-T and incubated with substrate (Tetramethyl benzidine, Zymed Laboratories Inc, Ca, USA) for 15 minutes, room temperature and protected from light. The reaction was stopped by adding 50 μl sulphuric acid (1.8 mol/L). The absorbance was measured at 450 nm in an ELISA reader (Spectra Max 250, Molecular Devices, Ca, USA).

The reference interval for the control group was: 0.03-0.15 (absorbancy values at 450 nm) and that of the patients was 0.23-0.34. It should be noted that all patients included in the study had an ELISA test value that was significantly elevated above the background values of the control group. This indicated presence of antiprostasome autoantibodies in all of the patients with PAD verified prostate cancer.

### REFERENCES

1. Nilsson B. O., Jin M. and Ronquist G. (1996) Immunolocalization of prostasomes in the human prostate. Upsala J Med Sci, 101:149-158.
2. Renneberg H., Konrad L., Dammshäuser I., Seitz J. and Aumüller G. (1997) Immunohistochemistry of prostasomes from human semen. The Prostate, 30:98-106.
3. Nilsson B. O., Jin M. and Ronquist G (1998) Monoclonal antibodies against human prostasomes. The Prostate, 35:178-184.
4. Schrimpf S., Hellman U., Carlsson L., Larsson A., Ronquist G., Nilsson B. O. (1999) Identification of dipeptidyl peptidase IV as the antigen of a monoclonal anti-prostasome antibody. The Prostate, 38:35-39.

1. An agent which selectively binds to anti-prostasome auto antibodies.

2. An agent according to claim 1, which is a diagnostic and/or prognostic reagent.

3. An agent according to claim 1, comprising structures involved in the binding of prostasomes or prostasomal-like material and anti-prostasome autoantibodies.

4. A kit for diagnosis/prognosis of prostasome-related diseases comprising a reagent according to claim 1.

5. An immunoassay for detection of anti-prostasome autoantibodies in body fluids comprising the reagent according to claim 2.

6. An immunoassay according to claim 5, wherein the body fluid is serum or plasma.

7. An immunoassay according to claim 5 wherein the agent is bound to a solid support.

8. An immunoassay according to claim 5, wherein the solid support is a microtitre plate.

9. An immunoassay according to claim 5 which is an ELISA or flow cytometry.

10. An in vitro method for diagnosing and/or prognosticating a condition reflecting the prostasome presence in body fluids, comprising:
a) binding of anti-prostasome autoantibodies with a component which selectively binds to anti-prostasome autoantibodies; and
b) detection of said binding.

11. A method according to claim 10, wherein said component in a) comprises structures involved in the binding of prostasomes or prostasomal-like material and anti-prostasome autoantibodies.

12. A method according to claim 10, wherein the condition is cancer.

13. A method according to claim 10, wherein the condition is prostasome-related disease.

14. A method according to claim 10, wherein the condition is prostate cancer.

15. A method according to claim 10, wherein the condition is prostatic disease.

16. A method according to claim 10, wherein the detection in step b) is by ELISA or flow cytometry techniques.

17. (canceled)

18. A method according to claim 11, wherein the condition is cancer.

19. A method according to claim 11, wherein the condition is prostasome-related disease.

20. A method according to claim 11, wherein the condition is prostate cancer.

21. A method according to claim 11, wherein the condition is prostatic disease.
